(19) United States
(12) Patent Application Publication
(10) Pub. No.: US 2008/0072640 A1
(43) Pub. Date: Mar. 27, 2008

(54) Insecticidal Fertilizer Solids and Method of Preparation

(76) Inventors: Kenneth E. Fersch, Apex, NC (US); Shannon M. Russell, Durham, NC (US)

Correspondence Address: Gerrett V. Davis;ROYLANCE, ABRAMS, BERDO & GOODMAN, L.L.P., Suite 600, 1300 19th Street, N.W., Washington, DC, 20036, US

(21) Appl. No.: 11/678,174

(22) Filed: Feb. 23, 2007

Related U.S. Application Data
(63) Continuation-in-part of application No. 11/525,876, filed on Sep. 25, 2006, now abandoned.

(51) Int. Cl. C05D 9/02; A61K 31/79

(52) U.S. Cl. 71/1; 424/78.24; 71/64.02

(57) ABSTRACT

An insecticidal fertilizer composition is prepared by contacting a solid fertilizer with a solution of a nicotinyl insecticide. The nicotinyl insecticide solution can be imidacloprid in a solvent, such as dimethyl sulfoxide, N-methyl-2-pyrrolidone or N-vinyl-2-pyrrolidone, and mixtures thereof. The solution contains at least about 10 wt % of the insecticide in solution to attain a solid, particulate fertilizer containing at least about 0.1 wt % of the insecticide.

### CROSS-REFERENCE TO RELATED APPLICATION

This application is a continuation-in-part application of prior application Ser. No. 11/525,876, filed Sep. 25, 2006, which is hereby incorporated by reference in its entirety.

### FIELD OF THE INVENTION

Insecticidal fertilizer solids are disclosed that contain an agrochemically effective plant fertilizer-containing solid that has been contacted with an insecticidally effective solution containing an effective amount of a nicotinyl insecticide. The insecticidal fertilizer is a solid fertilizer that contains an effective amount of the insecticide absorbed into or adsorbed on the solid fertilizer.

### BACKGROUND OF THE INVENTION

Insecticides that are agonists and antagonists of the nicotinergic acetylcholine receptors are disclosed in U.S. Pat. No. 4,742,060 (issued May 3, 1988). These insecticides have also been found useful as dermally applied insecticides. See, U.S. Pat. No. 6,232,328. These compounds are also very effective as systemic insecticides in plants. It has been found to be advantageous to co-apply these insecticides with an agrochemically effective fertilizer. One example is disclosed in U.S. Pat. No. 5,783,203.

U.S. Pat. No. 5,783,203 discloses a composition and processes for mixing solids that contain an agonist or antagonist of a nicotinergic acetylcholine receptor of insects (e.g., imidacloprid insecticide) with fertilizer solids. This is done by compressing or extruding the insecticidal solids with the fertilizer or attaching it to the fertilizer with a polymeric adhesive or inorganic adhesive (e.g., gypsum or cement).

The processes for achieving U.S. Pat. No. 5,783,203 are typically cumbersome and costly. As taught in that invention, numerous solids of different ingredients must be blended together. The production on a tablet, stick, granule requires a costly compression or extrusion process. Oversized and undersize product must each be separated and then recycled with granulation processes into subsequent batches.

A combining process that relies on external adhesion of insecticidal solids requires that the ingredients be milled to a very small particle size so that they will stay attached to the granule by the adhesive. In addition to a costly process step, milling to such a small particle size can create a dusty environment increasing the health risk to operators. Milling, compression, extrusion, and drying processes along with their support systems such as stream, chilled water, dust containment, etc. are capital intensive and have high operating costs.

The present inventors wondered whether a solvent could be found with high dissolving power for imidacloprid and chemically related nicotinyl insecticides, and whether an improved process could result. A solution of imidacloprid could be sprayed directly onto a fertilizer carrier and absorbed or adsorbed. This would be a very simple and cost effective process and would yield a product capable of very high nutrient values (>97% of the product as fertilizer).

The problem, as found by the inventors, is that fertilizer solids are able to absorb only very low levels of solubilized insecticide. Typically a fertilizer may only be able to absorb around 0.4-0.6 wt % of the insecticide solution. This might be increased to around 2 wt % by using drying agents such as diatomaceous earth or clay. Fortunately, imidacloprid is a very effective active pesticide wherein only 0.4 lb of active ingredient can be applied per acre per year under prevailing U.S. regulations. Combined with typical fertilizer application rates, this would amount to an applied concentration of imidacloprid in the composition that was in the range of about 0.1 to 0.5 wt % imidacloprid.

U.S. Pat. No. 5,783,203 mentions acetone as a solvent for imidacloprid. Acetone was found to have a solubility of less than 7 wt % imidacloprid. Using a 6 wt % solution imidacloprid in acetone would mean applying 1.67 wt % of the solution on the fertilizer based on the combined weight of the fertilizer and the solution to achieve a product with 0.10 wt % imidacloprid. This would be very difficult to achieve a dry product. Fertilizer products with 0.2 wt % and 0.5 wt % imidacloprid would require 3.33 wt % and 8.33 wt % respectively of the imidacloprid solution absorbed onto the fertilizer; concentrations which are not likely feasible in acetone. In addition, acetone is highly flammable which would make processing very difficult and expensive.

Accordingly, there is a continuing need in the industry for a process for producing a fertilizer composition.

### SUMMARY OF THE INVENTION

It is an object of the invention to provide a process for producing an effective, insecticidally active, fertilizer solid and to the resulting insecticidally active fertilizer.

It is a further object of the invention to provide an effective fertilizer composition for plants that includes an insecticidally effective nicotinyl component that is adsorbed or absorbed therein.

In accordance with these and other objects of the invention that will become apparent from the description herein, a solution of an insecticidally active compound is contacted with a solid fertilizer so that the fertilizer contains an effective amount of the insecticide.

The various aspects of the invention are basically attained by providing a solid fertilizer composition containing about 0.1 wt % to about 1.0 wt % insecticide based on the total weight of the fertilizer composition. In one embodiment, the fertilizer composition is substantially dry and is prepared without a drying step and without the use of drying agents, flow aids, adhesives or bonding agents.

The aspects of the invention are further attained by providing a process for producing a solid fertilizer composition by mixing or contacting a solid fertilizer with a solution comprising a nicotinyl insecticide. The solution is supplied in an amount to provide an effective amount of the insecticide in the fertilizer. In one embodiment, the solution is applied to the fertilizer in an amount of 0.5 wt % to about 3.0 wt % based on the weight of the solid fertilizer.

The aspects of the invention are also provided by forming a solution and contacting fertilizer solids with a solution containing at least about 10 wt % of an insecticide to produce a solid fertilizer composition containing about 0.1 wt % to about 1.0 wt % insecticide based on the weight of the composition. Preferred embodiments according to the invention include imidacloprid in either N-methyl-pyrrolidone or dimethyl sulfoxide. The solid is contacted with the fertilizer solids in an amount to avoid the need for an additional drying step, drying agents, flow aids, binders, or adhesives to physically associate the insecticide with the fertilizer.

Other aspects and advantages of the invention will become apparent from the following detailed description of the invention which disclose various embodiments of the invention.

### DETAILED DESCRIPTION OF THE INVENTION

The present invention is directed to a process for producing an insecticidal fertilizer and to the resulting insecticidal fertilizer. The invention is further directed to a solution containing an effective amount of an insecticide dissolved therein to be applied directly to a dry solid particulate fertilizer. Preferably, the solvent used in the solution is nonvolatile to provide a bioavailable coating for plant uptake without dust exposures during manufacture, packaging, transport or application.

The insecticidal fertilizer composition of the invention is preferably a solid, free-flowing composition that contains an insecticide, preferably a systemic insecticide, in an effective amount to treat the soil with the fertilizer composition while controlling the insect population. Preferably, the insecticide is included in an amount corresponding to the desired application rate of the fertilizer for a selected surface area so that the fertilizer composition can be applied at an effective rate for both the fertilizer and the insecticide.

Solvent System

The insecticidal solution of the invention contains a solvent system, preferably a nonvolatile solvent system, and a sufficient amount of the insecticide so that the amount of solution applied to the fertilizer solids provides effective concentration levels for both fertilizer and insecticide. It is also preferred that the solvation capacity of the solvent and its vapor pressure at 25° C. are selected so that the amount of insecticidal solution can be applied to the fertilizer solids without becoming wet or causing agglomeration of the solid particles.

The solvent system for the insecticide is preferably an organic solvent or mixture of organic solvents and cosolvents in which the insecticide is soluble in an amount of at least 10 wt %, preferably at least 15 wt %, and even more preferably at least 30 wt % for the applied insecticide at 25° C. Stated another way, the insecticide solution contains the insecticide in an amount within the range from at least about 90 g/l to about 1000 g/l of the solvent, preferably within the range of about 100-600 g/l, and even more preferably within the grange from about 150-350 g/l. Preferably, the insecticide is completely dissolved in the solution. In preferred embodiments, the solvent is nonvolatile and approved for use in agrochemical products and for human exposure.

In a preferred embodiment of the invention, the solvent system comprises a dimethyl sulfoxide or a pyrrolidone having the structure: N-R-2-pyrrolidone, where: R is an alkyl, alkenyl or hydroxy alkyl. The R moiety is typically an alkyl having 1-6 carbon atoms, an alkenyl having 1-6 carbon atoms or an hydroxyl alkyl having 1-6 carbon atoms. In one embodiment, R is selected from the group consisting of methyl, ethyl, hydroxyl ethyl, hexyl, hydroxy ethyl and vinyl. In still further embodiments, R can be octyl, decyl or dodecyl, although the solubility of the insecticide is lower than the other solvents noted herein. Such solvents include N-methyl-2-pyrrolidone, N-ethyl-2-pyrrolidone, N-butyl-2-pyrrolidone, N-hexyl-2-pyrrolidone, N-vinyl-2-pyrrolidone, and mixtures thereof. The N-vinyl-2-pyrrolidone can be amine-stabilized or caustic-stabilized.

Preferably, the solvent system for use in the present invention consists essentially of either N-methyl-2-pyrrolidone or DMSO. It has been found that imidacloprid is soluble in N-methyl-2-pyrrolidone at 25° C. in an amount of about 32 wt %. Amine-stabilized or caustic-stabilized N-vinyl-pyrrolidone can form a solution with imidacloprid in an amount of about 15 wt % at 25° C. Imidacloprid has been found to have a solubility in DMSO of about 32 wt % at room temperature of about 23° C. The solvent system can consist essentially of N-methyl-2-pyrrolidone, DMSO or a mixture of either N-methyl-2-pyrrolidone or DMSO with one or more co-solvents.

The solvent system of the present invention can be used with one or more stabilizing agents, suspending agents, thickeners, and similar formulation components to provide a pre-mixed, ready-to-use insecticidal solution or emulsion for use on fertilizer solids or other carrier solids. Suitable emulsifying and/or foam-forming agents that can be used in such a ready-to-use composition include non-ionic and anionic emulsifiers, such as polyoxyethylene-fatty acid esters, polyoxyethylene-fatty alcohol ethers, for example alkylaryl polyglycol ethers, alkyl sulphonates, alkyl sulphates, aryl sulphonates as well as albumin hydrolysis products. Suitable dispersing agents include lignin sulphite waste liquors and methylcellulose.

Suitable solid carriers include ground natural minerals, such as kaolin clay and other types of clays, talc, chalk, quartz, attapulgite, montmorillonite or diatomaceous earth, and ground synthetic minerals such as highly-dispersed silicic acid, alumina and silicates. As solid carriers for granules there may be used crushed and fractionated natural rocks such as calcite, marble, pumice, sepiolite and dolomite, as well as synthetic granules of inorganic and organic meals, and granules of organic material such as sawdust, coconut shells, corn cobs and tobacco stalks. See, U.S. Pat. No. 4,742,060 the disclosure of which is herein incorporated by reference.

The insecticidally effective active ingredient concentration in such ready-to-use forms can vary within wide limits. The active compound concentration of the use forms can be from 0.0000001 to 100% by weight of active compound, preferably between 0.0001 and 1% by weight.

The Insecticidally Effective Active Ingredient

The insecticide according to the invention is preferably a systemic insecticide. A particularly preferred systemic insecticide is an agonist or antagonist of nicotinergic acetylcholine receptors of insects, such as a nicotinyl insecticide. Nicotinyl insecticides that can be used are those as disclosed in U.S. Pat. No. 5,783,203 and U.S. Pat. No. 4,742,060, which are hereby incorporated by reference in their entirety. An especially preferred systemic insecticide is imidacloprid, 1-((6-chloro-3-pyridinyl)methyl)-N-nitro-2-imidazolidinimine.

The preferred insecticides have the following formula:

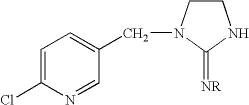

wherein R is NO₂ or CN.

A solution of the insecticide is prepared with a solvent system containing about 10 wt % to about 50 wt % of the insecticide, preferably about 10 wt % to about 35 wt %, and most preferably about 10 wt % to about 32 wt %. In another embodiment, the solution contains about 10 wt % to about 25 wt % of the insecticide and is particularly useful when applying a systemic insecticide such as imidacloprid. In further embodiments, the solution contains about 15 wt % to about 25 wt % of the insecticide in solution.

The Fertilizer Component

The fertilizer components can be organic or inorganic compounds. Examples of fertilizer components include coated and uncoated nitrogen-containing compounds such as urea, urea-formaldehyde condensation products, amino acids, ammonium salts and nitrates. Other fertilizer compounds include potassium salts such as potassium chloride, potassium sulfate, potassium nitrates, potassium and ammonium salts of phosphoric acid. The fertilizer can also contain nutrients such as manganese, magnesium, iron, boron, copper zinc, molybdenum, and cobalt. In other embodiments, the fertilizer can include phytohormones such as vitamin B₁ and indole-III-acetic acid.

The fertilizer solids are solid particles in the form of pellets, granules, flakes, and the like which may, or may not, have a controlled release coating, e.g., sulfur-coated urea. Commercially available fertilizers that are suitable include nitrogen, potassium, phosphorous and combinations thereof in various amounts as known in the art. Typical amounts of the components are about 0-30 wt % nitrogen compounds, 0-50 wt % phosphorous compounds and 0-50 wt % potassium compounds. A typical blend of these components might be something like 21-3-8 weight percent nitrogen, phosphorous pentoxide (P₂O₅) and potassium oxide (K₂O), respectively, and may include a combination of both coated and uncoated fertilizer solids.

Contact Methods

Many insecticides are sparingly soluble in organic solvents. As a result, excess amounts of the solution must be applied to the fertilizer in order to provide the fertilizer with an effective amount of the insecticide. As a result, a large amount of solvent is applied that causes the fertilizer to become wet. The wet fertilizer particles can agglomerate and foul equipment that effectively causing losses of the active pesticide and the agglomerated particles. To prevent agglomeration caused by large amounts of an insecticide solution, it is necessary to dry the fertilizer and/or use drying agents and highly volatile solvents that evaporate quickly. However, the use of volatile solvents raise environmental and other safety concerns. A drying step increases production time and expense.

One feature of the present invention is to provide a solvent system for an insecticide and particularly nicotinyl insecticides such that the insecticide is sufficiently soluble in the solvent system to be applied directly to fertilizer solids without the need for a drying step, drying agents, polymeric binders, polymer adhesives.

The process of the invention contacts the solid fertilizer solids with a solution of the insecticide by spraying, dripping or other suitable solid-liquid contact process. The insecticide is absorbed, adsorbed or coated on the fertilizer solids. The insecticide solution preferably contains at least 10 wt % of the insecticide in solution and the solution is contacted with the fertilizer in an amount of about 0.5 wt % to about 3.0 wt %, and typically in the amount of about 0.5 wt % to about 2.0 wt % based on the weight of the fertilizer. Applying the insecticide solution in these amounts provides an effective amount of the insecticide without the fertilizer solids becoming wet or subject to agglomeration. In a preferred embodiment, the insecticidal solution is substantially in the absence of adhesives or bonding agents for the insecticide.

The process of the invention applies an insecticidal compound or an insecticidal composition to a solid, particulate fertilizer in an amount that can be absorbed or adsorbed by the solid fertilizer. Preferably, the insecticide solution is applied to the particulate solid fertilizer in an effective amount that the insecticide can be absorbed, adsorbed or form a coating without agglomerating the particulate fertilizer solids and without causing the particulate fertilizer to become wet. The fertilizer is typically in the form of granules or prills having a particle size sufficient for application under conventional field conditions with existing equipment. The resulting insecticidal fertilizer composition contains the insecticide absorbed, adsorbed into the particles or as a coating on the surface of the particles.

The insecticide is contacted with or applied to the fertilizer particles in a manner to produce a free-flowing granular or particulate solid insecticidal fertilizer composition. Preferably, the process produces a substantially dry composition without the need for a drying step and without the need for added driers, flow aids or adhesives for the insecticide. The insecticidal solution is preferably contacted with the solid fertilizer by spraying, dripping, or similar dispersion techniques while the fertilizer solids are in motion (e.g., fluid bed, rotary mixer, tumbling, etc.) in batch or continuous operations.

The insecticidal solution can be applied to the fertilizer as a neat solution or an emulsified concentrate. Alternatively, the insecticidal solution can be microencapsulated to form solids that can be physically mixed, commingled with, or otherwise physically associated with the fertilizer solids as a free-flowing mixture or, if a binder or polymeric adhesive is used, as a combined particle.

Preferably, the insecticidal solution is contacted with the solid fertilizer to provide an effective amount of the insecticide without drying or the use of drying agents. In embodiments where higher amounts of the insecticide are desired or ambient conditions cause undesired hydration of the solids, a drying agent or flow aids such as diatomaceous earth can be added.

The insecticide concentration in the finished fertilizer is at least 0.1 wt % and typically in the range of about 0.1 wt % to about 1.0 wt % based on the weight of the insecticidal fertilizer composition. In one embodiment, the finished fertilizer contains about 0.1 wt % to about 0.5 wt % insecticide and preferably 0.2 wt % to about 0.5 wt % insecticide based on the weight of the insecticidal fertilizer composition.

### EXAMPLES

### Example 1

An imidacloprid solution was prepared by dissolving imidacloprid in dimethyl sulfoxide (DMSO). The solubility of imidacloprid in DMSO was found to be about 32 wt % at room temperature of about 23° C. The resulting solution can be sprayed onto a particulate fertilizer in an amount to provide a dry particulate composition containing about 0.1 wt % imidacloprid.

### Example 2

The solubility of imidacloprid TGAI in N-methyl-2-pyrrolidone (NMP) was investigated. NMP has a favorable flash point of 187° F. (closed cup), mild odor, and is slow to volatilize, which are all very favorable properties. Surprisingly, after all the solvents where tried, imidacloprid was soluble in NMP to almost 35 wt % at room temperature. A 30 wt % solution can easily be prepared to spray onto fertilizer solids. Spraying a 0.50 wt % solution on fertilizer would achieve 0.15 wt % active ingredient. Spraying a 0.67 wt % solution and 1.67% wt % solution would achieve 0.20 wt % and 0.50 wt % active ingredient, respectively. These are highly plausible concentrations for use.

### Example 3

In this experiment, a 29.5 wt % active imidacloprid solution in NMP was dripped onto a rolling bed of a 34-4-4 fertilizer solids to achieve 0.25 wt % active imidacloprid in the final product. This product was successfully made without the use of a drying aid though it had the appearance of being very close to the maximum amount that could be absorbed by this particular blend of fertilizer solids.

### Example 4

Example 4 was essentially a repeat of Example 3 except that the same solution was dripped onto a rolling bed of 34-3-4 fertilizer solids to achieve 0.51 wt % active imidacloprid in the final product. After 12 minutes of mixing, the product appeared wet. When 0.40 wt % of Diafil 110 (diatomaceous earth) was added, the formulation formed a dry, free flowing product.

While various embodiments have been chosen to illustrate the invention, it will be apparent that the various changes and modifications can be made without departing from the scope of the invention as defined in the appended claims.

What is claimed is:

1. A process for producing a fertilizer composition, said process comprising:
mixing fertilizer solids with an insecticidal solution including a nicotinyl insecticide in a solvent system comprising solvent system for said insecticide in which the insecticide is soluble in an amount of at least 10 wt % and is present in an amount sufficient, when applied to said fertilizer solids, to obtain a particulate fertilizer composition containing at least about 0.1 wt % of said nicotinyl insecticide based on the weight of the fertilizer composition.

2. The process of claim 1, wherein said nicotinyl insecticide is associated with said fertilizer by absorption, adsorption or as a coating.

3. The process of claim 1, wherein said solvent system comprises N-methyl-2-pyrrolidone or DMSO.

4. The process of claim 1, wherein said nicotinyl insecticide comprises imidacloprid.

5. The process of claim 1, wherein said solution contains about 10 wt % to about 35 wt % imidacloprid.

6. The process of claim 1, wherein said fertilizer composition comprises about 0.1 wt % to about 1 wt % imidacloprid.

7. The process of claim 1, wherein said solvent system comprises a pyrrolidone having the structure N-R-2-pyrrolidone, wherein: R is selected from the group consisting of methyl, ethyl, hydroxyethyl, hexyl, cyclohexyl, and vinyl.

8. The process of claim 1, wherein said solution consists essentially of DMSO and imidacloprid.

9. The process of claim 1, wherein the process is in the absence of a drying step and without the use of drying agents.

10. The process of claim 1, wherein the insecticide is applied as an emulsion.

11. A process for producing a particulate fertilizer composition, said process comprising:
associating an insecticidal solution comprising a solvent system and at least about 10 wt % of a nicotinyl insecticide with fertilizer solids in an amount sufficient to produce a fertilizer composition having at least about 0.1 wt % nicotinyl insecticide based on the weight of the fertilizer composition.

12. The process of claim 11, wherein said solvent system consists essentially of a pyrrolidone or dimethyl sulfoxide.

13. The process of claim 11, wherein said solvent system comprises dimethyl sulfoxide.

14. The process of claim 11, wherein said solvent comprises N-methyl-2-pyrrolidone.

15. The process of claim 11, wherein said solvent comprises a pyrrolidone derivative having the formula N-R-2-pyrrolidone, wherein R is selected from the group consisting of methyl, ethyl, hydroxyethyl, hexyl, cyclohexyl, and vinyl.

16. The process of claim 11, wherein said nicotinyl insecticide comprises imidacloprid, and the particulate fertilizer composition comprises about 0.1 wt % to about 0.5 wt % imidacloprid.

17. The process of claim 11, wherein said solution comprises about 10 wt % to about 35 wt % imidacloprid.

18. The process of claim 11, wherein said solution comprises imidacloprid in solution in an amount of at least about 35 g/l.

19. A process for producing a particulate, free-flowing insecticidal fertilizer composition, said process comprising:
applying to fertilizer solids an insecticidal solution comprising (a) an imidacloprid insecticide in (b) a solvent system that comprises a pyrrolidone or dimethyl sulfoxide in an amount sufficient to produce a fertilizer composition comprising at least about 0.1 wt % imidacloprid.

20. The process of claim 19, wherein said fertilizer composition comprises coated and uncoated fertilizer solids.

21. The process of claim 19, wherein said solution contains at least about 10 wt % imidacloprid.

22. The process of claim 19, wherein said solution contains about 10 wt % to about 35 wt % imidacloprid.

23. The process of claim 19, wherein said solution includes at least about 35 g/l imidacloprid in solution.

24. The process of claim 19, wherein the process comprises spraying said solution of imidacloprid onto moving fertilizer solids.

25. The process of claim 19, further comprising coating said fertilizer particles with said solution.

26. An insecticidal fertilizer composition comprising free-flowing fertilizer particles containing about 0.1 wt % to about 0.5 wt % imidacloprid in either N-methyl-pyrrolidone or dimethyl sulfoxide.

27. A ready-to-use insecticidal composition for coating fertilizer solids that comprises: (a) 90-1000 g/l of an agonist or antagonist of nicotinergic acetylcholine receptors of insects and (b) a liquid solvent system comprising N-methyl pyrrolidone or dimethyl sulfoxide.

28. A composition according to claim 27 that comprises: (a) 100-600 g/l of imidacloprid and (b) a liquid solvent system comprising N-methyl pyrrolidone.
